(19) United States
(12) Patent Application Publication
(10) Pub. No.: US 2018/0199987 A1
(43) Pub. Date: Jul. 19, 2018

(54) TREATMENT SYSTEM AND TREATMENT TOOL

(71) Applicant: OLYMPUS CORPORATION, Tokyo (JP)

(72) Inventor: Yuta SUGIYAMA, Tokyo (JP)

(73) Assignee: OLYMPUS CORPORATION, Tokyo (JP)

(21) Appl. No.: 15/918,437

(22) Filed: Mar. 12, 2018

Related U.S. Application Data
(63) Continuation of application No. PCT/JP2015/083292, filed on Nov. 26, 2015.

(51) Int. Cl. A61B 18/14

(52) U.S. Cl. CPC A61B 18/1445; A61B 2018/00791

(57) ABSTRACT

A treatment system includes: a probe including an energy generator provided in the probe and configured to generate energy, and a treatment surface provided at a distal end of the probe and configured to apply the energy to a living tissue; and a controller including a hardware, the controller being configured to: acquire a temperature of an outer surface of the probe other than the treatment surface; sequentially store temperatures of the energy generator in association with times when the temperatures of the energy generator are acquired; store a plurality of weighting factors calculated by experiment in advance in association with times going back from a current time to a past time; and multiply the sequentially stored temperatures of the energy generator and the plurality of weighting factors for the corresponding times, perform integration, and estimate the temperature of the outer surface.

### CROSS-REFERENCE TO RELATED APPLICATION

This application is a continuation of International Application No. PCT/JP2015/083292, filed on Nov. 26, 2015, the entire contents of which are incorporated herein by reference.

### BACKGROUND

The present disclosure relates to a treatment system and a treatment tool.

In the related art, there has been known a treatment system for treatment (joining (or anastomosis), dissection, and the like) of a living tissue by applying energy to the living tissue (refer to, for example, JP 2012-24583 A).

A treatment system (thermal tissue surgery system) disclosed in JP 2012-24583 A includes a pair of jaws that is supported so as to be openable and closable and an energy source that supplies electric power to heat generating resistance elements embedded in the pair of jaws. Then, in the treatment system, a living tissue is grasped by the pair of jaws, and electric power is supplied to each heat generating resistance element, so that each heat generating resistance element and the living tissue are heated to treat the living tissue.

### SUMMARY

A treatment system according to one aspect of the present disclosure may include: a probe including an energy generator provided in the probe and configured to generate energy, and a treatment surface provided at a distal end of the probe and configured to apply the energy to a living tissue; and a controller including a hardware, the controller being configured to: acquire a temperature of an outer surface of the probe other than the treatment surface; sequentially store temperatures of the energy generator in association with times when the temperatures of the energy generator are acquired; store a plurality of weighting factors calculated by experiment in advance in association with times going back from a current time to a past time; and multiply the temperatures of the energy generator sequentially and the plurality of weighting factors for the corresponding times, perform integration, and estimate the temperature of the outer surface.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating a treatment system according to a first embodiment;

FIG. 2 is an enlarged view of a distal end (treatment unit) of a treatment tool illustrated in FIG. 1;

FIG. 3 is a view illustrating a first holding member and an energy generator illustrated in FIG. 2;

FIG. 4 is a view illustrating the first holding member and the energy generator illustrated in FIG. 2;

FIG. 5 is a view illustrating the first holding member and the energy generator illustrated in FIG. 2;

FIG. 6 is a block diagram illustrating a control device illustrated in FIG. 1;

FIG. 7 is a flowchart illustrating operations of the control device illustrated in FIG. 6;

FIG. 8 is a block diagram illustrating a control device constituting a treatment system according to a second embodiment;

FIG. 9 is a flowchart illustrating operations of the control device illustrated in FIG. 8;

FIG. 10 is a diagram illustrating a calculation example of Step S17 illustrated in FIG. 9;

FIG. 11 is a diagram illustrating an example of weighting factors used in Steps S16 and S17 illustrated in FIG. 9;

FIG. 12 is a block diagram illustrating a control device constituting a treatment system according to a third embodiment;

FIG. 13 is a diagram illustrating an example of an analysis model used in a temperature estimation unit illustrated in FIG. 12; and

FIG. 14 is a flowchart illustrating operations of the control device illustrated in FIG. 12.

### DETAILED DESCRIPTION

Hereinafter, embodiments will be described with reference to the drawings. In addition, the present disclosure is not limited by the embodiments described below. In addition, in the description of the drawings, the same components are denoted by the same reference numerals.

### First Embodiment

Schematic Configuration of Energy Treatment System

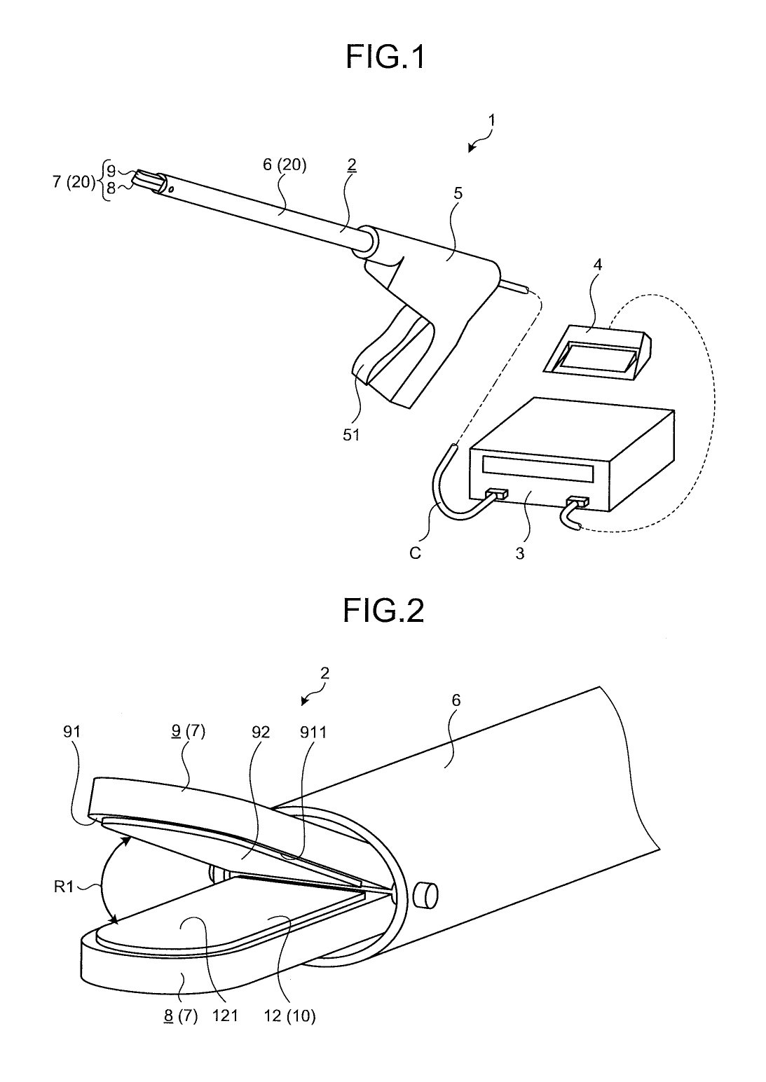

FIG. 1 is a diagram schematically illustrating a treatment system 1 according to a first embodiment.

The treatment system 1 treats (joins (or anastomoses), dissects, and the like) a living tissue by applying energy (thermal energy in the first embodiment) to the living tissue to be treated. As illustrated in FIG. 1, the treatment system 1 includes a treatment tool 2, a control device 3, and a foot switch 4.

Structure of Treatment Tool

The treatment tool 2 is, for example, a linear type surgical medical treatment tool for performing treatment on a living tissue through an abdominal wall. As illustrated in FIG. 1, the treatment tool 2 includes a handle 5, a shaft 6, and a treatment unit 7.

The shaft 6 and the treatment unit 7 have a function as a probe 20 (FIG. 1) according to the present disclosure.

The handle 5 is a component to be gripped by an operator. As illustrated in FIG. 1, the handle 5 is provided with a manipulation knob 51.

As illustrated in FIG. 1, the shaft 6 has a substantially cylindrical shape, and one end (the right end in FIG. 1) thereof is connected to the handle 5. The treatment unit 7 is attached to the other end (the left end in FIG. 1) of the shaft 6. An opening/closing mechanism (not illustrated) opening and closing the first and second holding members 8 and 9 (FIG. 1) constituting the treatment unit 7 in response to manipulations of the manipulation knob 51 by the operator is provided inside the shaft 6. In addition, the treatment unit 7 is not limited to the configuration where the first and second holding members 8 and 9 are opened and closed, and the treatment unit may have a pen-shaped configuration. In addition, inside the shaft 6, an electric cable C (FIG. 1) connected to the control device 3 is arranged from one end side (the right end side in FIG. 1) to the other end side (the left end side in FIG. 1) through the handle 5.

Configuration of Treatment Unit

FIG. 2 is an enlarged view of the distal end (treatment unit 7) of the treatment tool 2.

The treatment unit 7 is a component that grasps the living tissue and treats the living tissue. As illustrated in FIG. 1 or FIG. 2, the treatment unit 7 includes first and second holding members 8 and 9.

The first and second holding members 8 and 9 are pivoted at the other end (the left end in FIG. 1 and FIG. 2) of the shaft 6 so as to be openable and closable in the direction of an arrow R1 (FIG. 2), so that the living tissue may be grasped in response to manipulations of the manipulation knob 51 by the operator.

Hereinafter, the configurations of the first and second holding members 8 and 9 will be described in order.

Configuration of First Holding Member

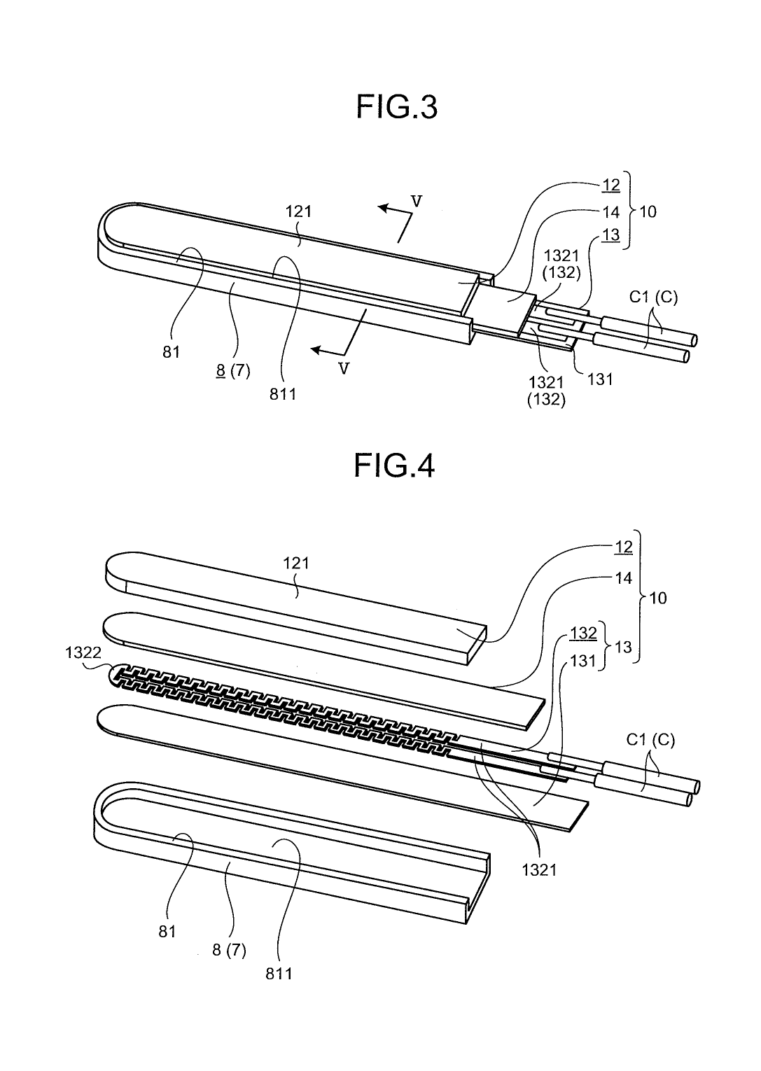

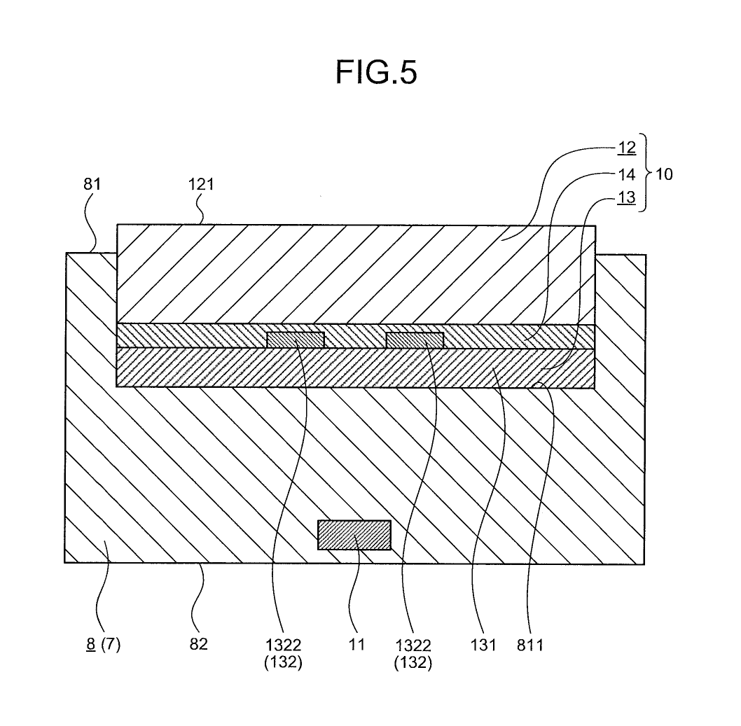

FIGS. 3 to 5 are views illustrating the first holding member 8 and an energy generator 10. Specifically, FIG. 3 is a perspective view of the first holding member 8 and the energy generator 10 as viewed from the upper side in FIGS. 1 and 2. FIG. 4 is an exploded perspective view of FIG. 3. FIG. 5 is a cross-sectional view taken along the line V-V of FIG. 3.

The first holding member 8 is arranged on the lower side in FIGS. 1 and 2 relative to the second holding member 9 and has a substantially rectangular parallelepiped shape extending along the central axis of the shaft 6. The upper side surface of the first holding member 8 in FIGS. 1 to 5 functions as a first grasping surface 81 that grasps the living tissue with the second holding member 9.

A first recessed portion 811 that is recessed downward in FIGS. 4 and 5 and extends from one end (the right end in FIG. 4) of the first holding member 8 toward the other end side along the longitudinal direction of the first holding member 8 is arranged at a substantially central position in the width direction of the first grasping surface 81.

As illustrated in FIGS. 2 to 5, the first recessed portion 811 is a portion where the energy generator 10 is provided.

The first holding member 8 described above is formed by molding a resin material (for example, fluororesin or the like). When the first holding member 8 is molded, a temperature sensor 11 (FIG. 5) is sealed near the outer surface of the first holding member 8 other than the first grasping surface 81.

The temperature sensor 11 is configured with a thermistor or the like, and detects the temperature of the outer surface of the first holding member 8. Then, the temperature sensor 11 is electrically connected to the electric cable C rotated to the other end of the shaft 6 and outputs a signal corresponding to the detected temperature (hereinafter, referred to as the outer surface temperature) to the control device 3. That is, the temperature sensor 11 has a function as a temperature acquisition unit and a controller 33 according to the present disclosure.

The arranged position of the temperature sensor 11 may be any position as long as the position is near the outer surface of the probe 20 other than a treatment surface 121. In the first embodiment, the temperature sensor 11 is, for example, arranged near the outer surface (at nearly the center position in the width direction in a back surface 82) having the highest temperature on the back surface 82 (FIG. 5) side facing the first grasping surface 81 in the outer surface of the first holding member 8. In the first embodiment, the temperature sensor 11 is arranged near the outer surface, but the present disclosure is not limited thereto, and the temperature sensor 11 may be arranged in the state of being exposed to the outer surface.

The energy generator 10 generates energy (thermal energy in the first embodiment) under the control of the control device 3. As illustrated in FIGS. 3 to 5, the energy generator 10 includes a flexible board 13, and an adhesive sheet 14.

The heat transfer plate 12 is a thin plate having an elongated shape (an elongated shape extending in the longitudinal direction (the left-right direction in FIGS. 3 and 4) of the first holding member 8) and being made of a material such as copper. Then, in the state where the living tissue is grasped by the first and second holding members 8 and 9, the surface 121 (the surface on the upper side in FIGS. 2 to 5) of the heat transfer plate 12 is in contact with the living tissue to transfer the heat from the flexible board 13 to the living tissue (apply thermal energy to the living tissue).

The surface 121 has a function as a treatment surface according to the present disclosure in order to apply thermal energy to the living tissue. Hereinafter, for the convenience of description, the surface 121 will be referred to as the treatment surface 121.

A portion of the flexible board 13 generates heat and functions as a sheet heater that heats the heat transfer plate 12 by the generated heat. As illustrated in FIGS. 3 to 5, the flexible board 13 includes an insulating board 131 and a wiring pattern 132.

The insulating board 131 is a sheet having an elongated shape (an elongated shape extending in the longitudinal direction of the first holding member 8 (in the left-right direction in FIGS. 3 and 4)) and being made of polyimide which is an insulating material.

In addition, the material of the insulating board 131 is not limited to polyimide, and for example, a high heat resistant insulating material such as aluminum nitride, alumina, glass, or zirconia may be adopted.

Herein, the width dimension of the insulating board 131 is set to be substantially equal to the width dimension of the heat transfer plate 12. In addition, the length dimension (the length dimension in the left-right direction in FIGS. 3 and 4) of the insulating board 131 is set to be longer than the length dimension of the heat transfer plate (the length dimension in the left-right direction in FIGS. 3 and 4).

As illustrated in FIGS. 3 to 5, the wiring pattern 132 is formed by processing stainless steel (SUS 304) which is a conductive material and includes a pair of lead wire connection portions 1321 (FIGS. 3 and 4) and a heat generation pattern 1322 (FIGS. 4 and 5). The wiring pattern 132 is bonded to one surface of the insulating board 131 by thermocompression bonding.

In addition, the material of the wiring pattern 132 is not limited to stainless steel (SUS 304), but other stainless material (for example, 400 series) may be used, or a conductive material such as platinum or tungsten may be adopted. In addition, the wiring pattern 132 is not limited to a configuration where the wiring pattern 132 is bonded to one surface of the insulating board 131 by thermocompression bonding, but a configuration formed on the one surface by vapor deposition or the like may be adopted.

As illustrated in FIG. 3 or FIG. 4, the pair of lead wire connection portions 1321 is provided so as to face each other along the width direction of the insulating board 131 and is joined (connected) to two lead wires C1 constituting the electric cable C.

One end of the heat generation pattern 1322 is connected (electrically conducted) to one of the lead wire connection portions 1321, and the heat generation pattern extends from the one end along a U shape that follows the outer edge shape of the insulating board 131 while meandering in a wavelike manner, and the other end thereof is connected (electrically conducted) to the other lead wire connection portion 1321.

Then, the heat generation pattern 1322 generates heat by applying (electrically conducting) the voltage to the pair of lead wire connection portions 1321 by the control device 3 through the two lead wires C1.

As illustrated in FIGS. 3 to 5, the adhesive sheet 14 is interposed between the heat transfer plate 12 and the flexible board 13, and in the state where a portion of the flexible board 13 protrudes from the heat transfer plate 12, the back surface (the surface on the side opposite to the treatment surface 121) of the heat transfer plate 12 and the one surface (the surface on the wiring pattern 132 side) of the flexible board 13 are attached and fixed. The adhesive sheet 14 is a sheet having an elongated shape (an elongated shape in the longitudinal direction (the left-right direction in FIGS. 3 and 4) of the first holding member 8), having excellent heat conductivity and insulating property, being resistant to high temperature, having adhesion property and is formed by mixing a high heat conductive filler (nonconductive material) such as alumina, boron nitride, graphite, or aluminum nitride with a resin such as epoxy or polyurethane.

Herein, the width dimension of the adhesive sheet 14 is set to be substantially equal to the width dimension of the insulating board 131. In addition, the length dimension (the length dimension in the left-right direction in FIGS. 3 and 4) of the adhesive sheet 14 is set so as to be longer than the length dimension (the length dimension in the left-right direction in FIGS. 3 and 4) of the heat transfer plate 12 and to be shorter than the length dimension (the length dimension in the left-right direction in FIGS. 3 and 4) of the insulating board 131.

The heat transfer plate 12 is arranged so as to cover the entire region of the heat generation pattern 1322. In addition, the adhesive sheet 14 is arranged so as to cover the entire region of the heat generation pattern 1322 and cover a portion of the pair of lead wire connection portions 1321. That is, the adhesive sheet 14 is arranged in the state where the adhesive sheet protrudes to the right side in FIGS. 3 and 4 with respect to the heat transfer plate 12. Then, the two lead wires C1 (FIGS. 3 and 4) are respectively joined (connected) to a region (a region not covered with the adhesive sheet 14) exposed to the outside of the pair of lead wire connection portions 1321.

Configuration of Second Holding Member

As illustrated in FIG. 2, the second holding member 9 has substantially the same outer shape as the first holding member 8. The lower side surface of the second holding member 9 in FIG. 2 functions as a second grasping surface 91 that grasps the living tissue with the first holding member 8.

Similarly to the first holding member 8, at nearly the center position in the width direction of the second grasping surface 91, provided is a second recessed portion 911 that is recessed upward in FIG. 2 and extends from one end (the right end in FIG. 2) of the second holding member 9 toward the other end side along the longitudinal direction of the second holding member 9.

As illustrated in FIG. 2, the second recessed portion 911 is a portion where a heat transfer plate 92 similar to the heat transfer plate 12 is provided.

Configuration of Control Device and Foot Switch

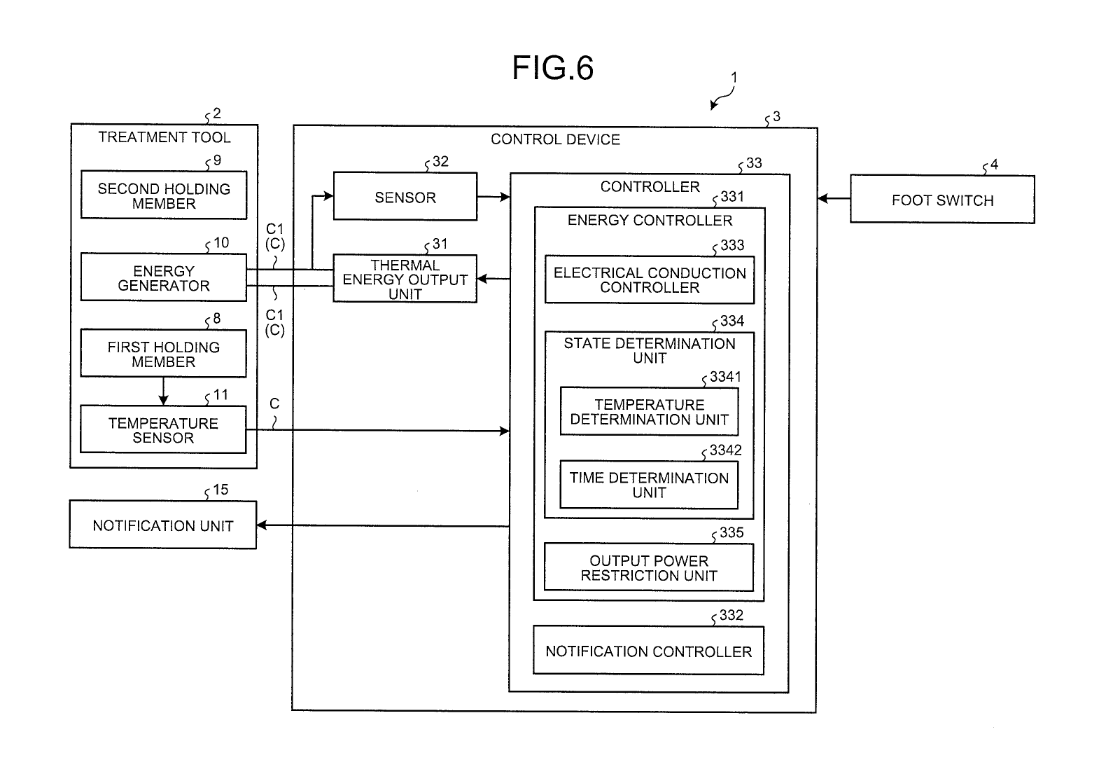

FIG. 6 is a block diagram illustrating the control device 3.

In FIG. 6, the main components are mainly illustrated as the configuration of the treatment system 1 (control device 3).

The foot switch 4 receives a first user operation shifting the treatment tool 2 from the standby state (state of waiting for the treatment of the living tissue) to the treatment state (state of treating the living tissue) when the foot switch is pressed (turned on) by the operator's foot. In addition, the foot switch 4 receives a second user operation shifting the treatment tool 2 from the treatment state to the standby state when the operator's foot is released (turned off) from the foot switch 4. Then, the foot switch 4 outputs signals corresponding to the first and second user manipulations to the control device 3.

In addition, the configuration for receiving the first and second user manipulations is not limited to the foot switch 4, and other manually operated switches or the like may be adopted.

The control device 3 totally controls the operations of the treatment tool 2. As illustrated in FIG. 6, the control device 3 includes a thermal energy output unit 31, a sensor 32, and a controller 33.

Under the control of the controller 33, the thermal energy output unit 31 applies (electrically conducts) the voltage to the energy generator 10 (the wiring pattern 132) through the two lead wires C1.

The sensor 32 detects the current value and the voltage value supplied (electrically conducted) from the thermal energy output unit 31 to the energy generator 10. Then, the sensor 32 outputs signals corresponding to the detected current value and voltage value to the controller 33.

The controller 33 includes a central processing unit (CPU) and the like, and performs feedback control of the energy generator 10 (wiring pattern 132) according to a predetermined control program. As illustrated in FIG. 6, the controller 33 includes an energy controller 331 and a notification controller 332.

The energy controller 331 controls an output value (power value) supplied (electrically conducted) to the energy generator 10. As illustrated in FIG. 6, the energy controller 331 includes an electrical conduction controller 333, a state determination unit 334, and an output power restriction unit 335.

When the foot switch 4 is turned on (when the foot switch 4 receives the first user operation), the electrical conduction controller 333 shifts the treatment tool 2 to the treatment state.

Specifically, when the treatment tool 2 is shifted to the treatment state, while monitoring the temperature of the energy generator 10, the electrical conduction controller 333 supplies, to the energy generator 10 (the wiring pattern 132), the output value (power value) necessary for maintaining the energy generator 10 to the target temperature through the thermal energy output unit 31 (performs the feedback control of the energy generator 10).

In the first embodiment, the following temperature is adopted as the temperature of the energy generator 10 used in the feedback control.

That is, based on the current value and voltage value detected by the sensor 32 (current value and voltage value supplied (electrically conducted) from the thermal energy output unit 31 to the energy generator 10 (wiring pattern 132)), a resistance value of the wiring pattern 132 is obtained. Then, the resistance value of the wiring pattern 132 is converted into a temperature, and the converted temperature is defined as a temperature of the energy generator 10 (hereinafter, referred to as a heater temperature).

The temperature of the energy generator 10 used in the feedback control is not limited to the heater temperature described above. For example, a temperature sensor configured with a thermocouple, a thermistor, or the like may be provided on the heat transfer plate 12 or the like, and the temperature detected by the temperature sensor may be used as a temperature of the energy generator 10.

In addition, when the foot switch 4 is turned off (when the foot switch 4 receives the second user operation), the electrical conduction controller 333 shifts the treatment tool 2 to the standby state.

Specifically, when the treatment tool 2 is shifted to the standby state, the electrical conduction controller 333 supplies a minimum output power (for example, 0.1 W) to the energy generator 10 (wiring pattern 132) through the thermal energy output unit 31 so that the heater temperature may be acquired (the current value and the voltage value may be detected by the sensor 32).

The state determination unit 334 determines the state of the outer surface of the first holding member 8 based on the outer surface temperature detected by the temperature sensor 11. As illustrated in FIG. 6, the state determination unit 334 includes a temperature determination unit 3341 and a time determination unit 3342.

The temperature determination unit 3341 compares the outer surface temperature detected by the temperature sensor 11 with a predetermined temperature restriction value (corresponding to a threshold value according to the present disclosure, for example, 80° C.) and determines whether or not the outer surface temperature is equal to or higher than the temperature restriction value.

When it is determined by the temperature determination unit 3341 that the outer surface temperature is equal to or higher than the temperature restriction value, the time determination unit 3342 sets a timer (initial value is 0) to a predetermined time (for example, 3 seconds (hereinafter, seconds is referred to as “s”)). In addition, when it is determined by the temperature determination unit 3341 that the outer surface temperature is lower than the temperature restriction value, the time determination unit 3342 counts down the timer and determines whether or not the value of the timer is equal to or lower than 0.

Based on the determination result of the state determination unit 334, the output power restriction unit 335 restricts (restricts the output) the output value (power value) to be supplied (electrically conducted) to the energy generator 10 (wiring pattern 132) (restricts the amount of energy generated in the energy generator 10).

The notification controller 332 controls operations of a notification unit 15 based on the determination result of the state determination unit 334.

In the first embodiment, the notification unit 15 includes a speaker notifying predetermined information (generating a warning sound) by voice. The notification unit 15 is not limited to the speaker, and may be a display displaying predetermined information, a light emitting diode (LED) notifying predetermined information by lighting or blinking, or the like.

Operation of Control Device

Next, operations of the control device 3 described above will be described.

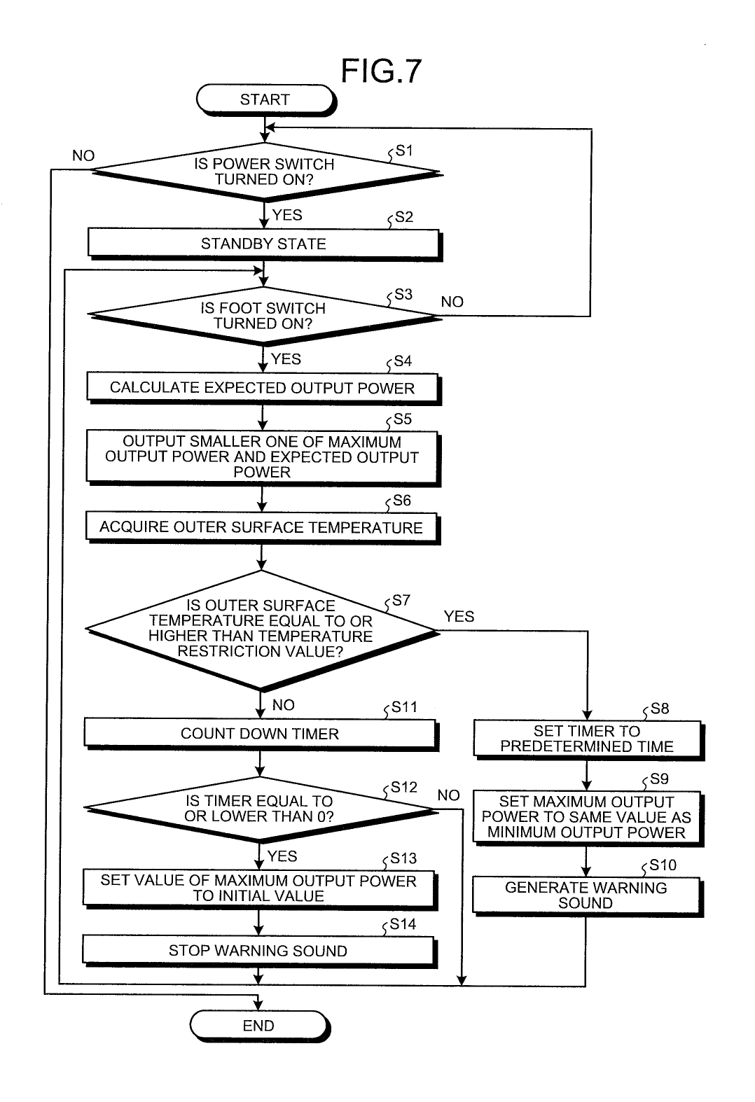

FIG. 7 is a flowchart illustrating the operations of the control device 3.

After the operator turns on the power switch (not illustrated) of the treatment system 1 (control device 3) (Step S1: Yes), the electrical conduction controller 333 shifts the treatment tool 2 to the standby state (Step S2).

Specifically, in Step S2, the electrical conduction controller 333 supplies (electrically conducts) a minimum output power (for example, 0.1 W) to the energy generator 10 through the thermal energy output unit 31. That is, in this state, the heater temperature may be acquired (the current value and the voltage value may be detected by the sensor 32).

After Step S2, the controller 33 determines whether or not the foot switch 4 is turned on (Step S3).

When it is determined that the foot switch 4 is turned off (or the OFF state is continued) (Step S3: No), the control device 3 returns to Step S1.

On the other hand, when it is determined that the foot switch 4 is turned on (Step S3: Yes), the electrical conduction controller 333 shifts the treatment tool 2 to the treatment state (Steps S4 and S5).

Specifically, in Step S4, the electrical conduction controller 333 calculates an output value (expected output power) necessary for setting the energy generator 10 to the target temperature while monitoring the heater temperature. Then, in Step S5, the electrical conduction controller 333 supplies (electrically conducts) the smaller one of the expected output power and a maximum output power (for example, the initial value is 100 W) to the energy generator 10 through the thermal energy output unit 31).

After Step S5, the temperature determination unit 3341 acquires the outer surface temperature detected by the temperature sensor 11 (Step S6).

After Step S6, the temperature determination unit 3341 compares the outer surface temperature with the temperature restriction value and determines whether or not the outer surface temperature is equal to or higher than the temperature restriction value (Step S7).

When it is determined that the outer surface temperature is equal to or higher than the temperature restriction value (Step S7: Yes), the time determination unit 3342 sets the timer to a predetermined time (for example, 3 s) (Step S8).

After Step S8, the output power restriction unit 335 sets the maximum output power (for example, the initial value is 100 W) to the same value as the minimum output power (for example, 0.1 W) (Step S9).

As described above, when the treatment tool 2 is shifted to the treatment state, in Step S5, the smaller one of the expected output power and the maximum output power is supplied (electrically conducted) to the energy generator 10. Therefore, in Step S9, the output power restriction unit 335 restricts (output-restricts) the output value (power value) supplied (electrically conducted) to the energy generator 10 to the minimum output power (for example, 0.1 W) by setting the maximum output power (for example, the initial value is 100 W) to the minimum output power (for example, 0.1 W).

After Step S9, the notification controller 332 operates the notification unit 15 to generate a warning sound (Step S10). After that, the control device 3 returns to Step S3.

On the other hand, when it is determined that the outer surface temperature is lower than the temperature restriction value (Step S7: No), the time determination unit 3342 counts down the timer (Step S11).

Specifically, when the timer has the initial value of 0, the time determination unit 3342 counts down the timer in Step S11 to set the timer to a minus value. In addition, after setting the timer to the predetermined time (for example, 3 s), the time determination unit 3342 counts down the timer from the predetermined time in Step S11.

After Step S11, the time determination unit 3342 determines whether or not the timer is equal to or lower than 0 (Step S12).

When it is determined that the timer is not equal to or lower than 0 (Step S12: No), the control device 3 returns to Step S3.

On the other hand, when it is determined that the timer is equal to or lower than 0 (Step S12: Yes), the output power restriction unit 335 sets the maximum output power to the initial value (for example, 100 W) (Step S13).

That is, when the output power restriction is performed in Step S9, the output power restriction unit 335 releases the output power restriction in Step S13. In addition, when the output power restriction is not performed in Step S9, the maximum output power is continued to be set to the initial value in Step S13.

After Step S13, the notification controller 332 stops the operations of the notification unit 15 and stops the warning sound (Step S14). After that, the control device 3 returns to Step S3.

That is, when the warning sound is generated in Step S10, the notification controller 332 stops the warning sound in Step S14. When no warning sound is generated in Step S10, the state where the warning sound is stopped is continued in Step S14.

The treatment tool 2 according to the first embodiment described above includes the temperature sensor 11 that detects the outer surface temperature and outputs a signal corresponding to the detected outer surface temperature to the control device 3.

Therefore, when the outer surface temperature is equal to or higher than the temperature restriction value, based on the outer surface temperature detected by the temperature sensor 11, the control device 3 may perform output power restriction (Step S9) and generation of a warning sound (Step S10). In other words, when the outer surface temperature becomes high, the outer surface temperature may be reduced by the output power restriction, and the generation of the warning sound notifies the operator that the outer surface temperature has become high.

Therefore, according to the treatment tool 2 of the first embodiment, it is possible to obtain an effect of avoiding unintentional actions on the living tissue at portions other than the treatment surface 121 of the first holding member 8.

### Second Embodiment

Next, a second embodiment will be described.

In the following description, the same configurations as those of the first embodiment described above are denoted by the same reference numerals, and the detailed description thereof will be omitted or simplified.

In the treatment system 1 according to the first embodiment described above, the outer surface temperature is actually measured by the temperature sensor 11.

In contrast, in a treatment system according to the second embodiment, the temperature sensor 11 is omitted, and the outer surface temperature is estimated based on the temperature of the energy generator and an assumed atmosphere temperature outside the treatment system.

Hereinafter, the configuration of the treatment system according to the second embodiment and the operations of the control device constituting the treatment system will be sequentially described.

Configuration of Treatment System

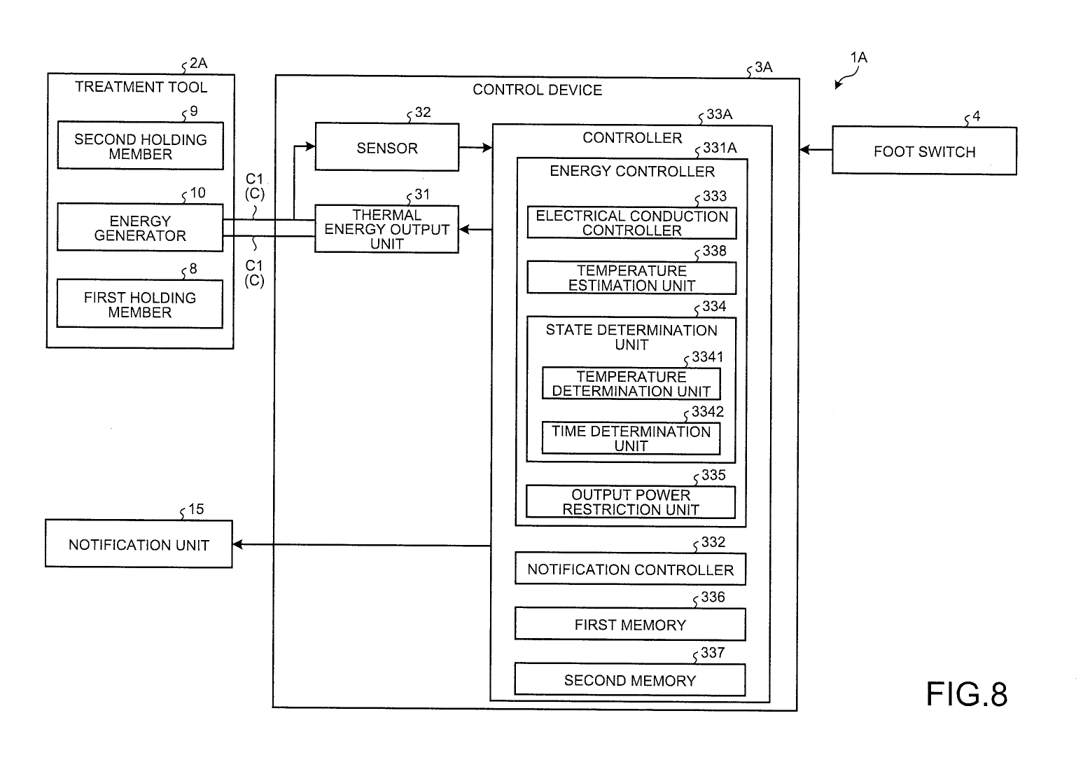

FIG. 8 is a block diagram illustrating a control device 3A constituting a treatment system 1A according to the second embodiment.

As illustrated in FIG. 8, the treatment system 1A adopts a treatment tool 2A in which the temperature sensor 11 is omitted instead of the treatment tool 2 with respect to the treatment system 1 (FIG. 6) described in the first embodiment and adopts a control device 3A (controller 33A) in which a portion of functions is added to the controller 33 instead of the control device 3.

As illustrated in FIG. 8, the controller 33A includes first and second memories 336 and 337 added to the controller 33 (FIG. 6) described in the first embodiment and adopts an energy controller 331A in which a temperature estimation unit 338 is added to the energy controller 331.

The first memory 336 sequentially stores the heater temperatures calculated at predetermined sampling intervals (for example, 0.05 s) by the energy controller 331A (electrical conduction controller 333) based on the current value and the voltage value detected by the sensor 32 in association with the time when the heater temperature is calculated. That is, the first memory 336 has a function as a first storage unit according to the present disclosure.

Herein, the first memory 336 stores only the heater temperatures calculated for a predetermined time (the time equal to an integration time described below) sequentially from the present to the past. That is, when a new heater temperature is calculated and the latest heater temperature is stored in the first memory 336, the oldest heater temperature is erased.

The second memory 337 is configured with a nonvolatile memory and stores a control program executed by the controller 33A and an assumed atmosphere temperature outside the treatment system 1A (assumed to be used in a living body, and thus, about 37° C. to about 40° C.). In addition, the second memory 337 stores a plurality of weighting factors calculated by experiment in advance in association with the time going back to the past from the present. That is, the second memory 337 has functions as second and third storage units according to the present disclosure.

A method of calculating a plurality of weighting factors will be described later.

The temperature estimation unit 338 has a function as a temperature acquisition unit according to the present disclosure and estimates the outer surface temperature based on the information stored in the first and second memories 336 and 337.

Operation of Control Device

Next, the operations of the control device 3A described above will be described.

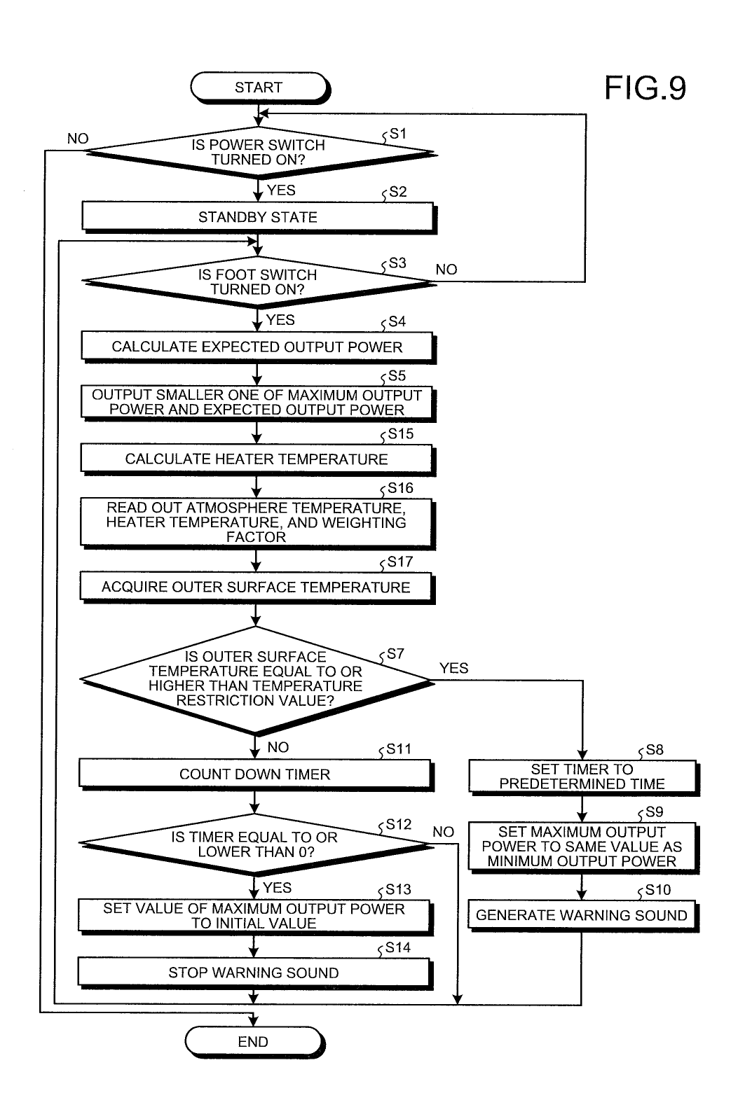

FIG. 9 is a flowchart illustrating the operations of the control device 3A.

As illustrated in FIG. 9, the operations of the control device 3A according to the second embodiment are different from the operations (FIG. 7) of the control device 3 described in the first embodiment only in that Steps S15 to S17 are added instead of Step S6. Therefore, only Steps S15 to S17 will be described below.

Step S15 is executed after Step S5.

Specifically, in Step S15, the electrical conduction controller 333 calculates the heater temperature based on the current value and the voltage value detected by the sensor 32. Then, the electrical conduction controller 333 stores the calculated heater temperature in the first memory 336.

After Step S15, the temperature estimation unit 338 reads out the atmosphere temperature, the heater temperature, and the weighting factor from the first and second memories 336 and 337 (Step S16).

After Step S16, the temperature estimation unit 338 inserts the read atmosphere temperature, the heater temperature, and the weighting factor into the following Mathematical Formula (1) to calculate (estimate) the outer surface temperature (Step S17). After that, the control device 3A proceeds to Step S7.

$${T}_{surface} = {\sum }_{t = 0}^{Period \_ ma x} \{ α ( t ) ( {T}_{heater} ( t ) - {T}_{atmospher} ) Δ t \} + {T}_{atmosphere} \qquad ( 1 )$$

Herein, in Mathematical Formula (1), T_{surface} is an outer surface temperature to be calculated (estimated). Period_max is an integration time. t is a time going back to the past from the current time (the time t at the current time is 0 s, a time t before the current time is a negative value). α(t) is a weighting factor with respect to a time t that goes back to the past from the current time. Tₕₑₐₜₑᵣ(t) is a heater temperature with respect to a time t that goes back to the past from the current time. Tₐₜₘₒₛₚₕₑᵣₑ is an assumed atmosphere temperature outside the treatment system 1A. At is a sampling interval (for example, 0.05 s).

Calculation Example of Outer Surface Temperature T_{surface}

Next, a calculation example of the outer surface temperature T_{surface} in Step S17 described above will be described.

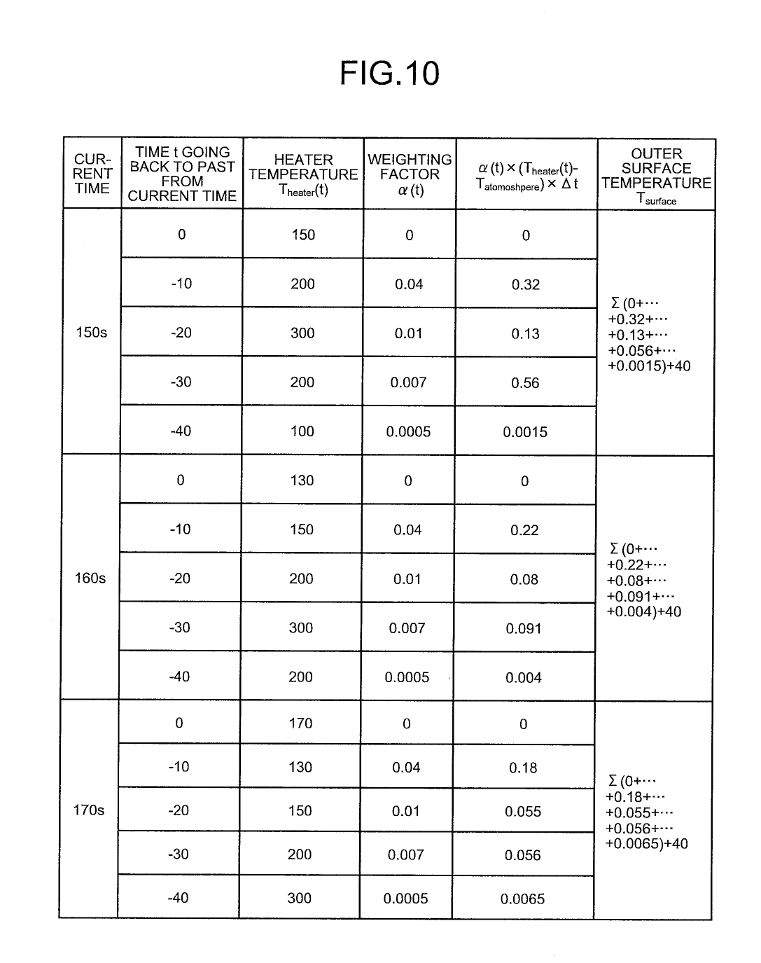

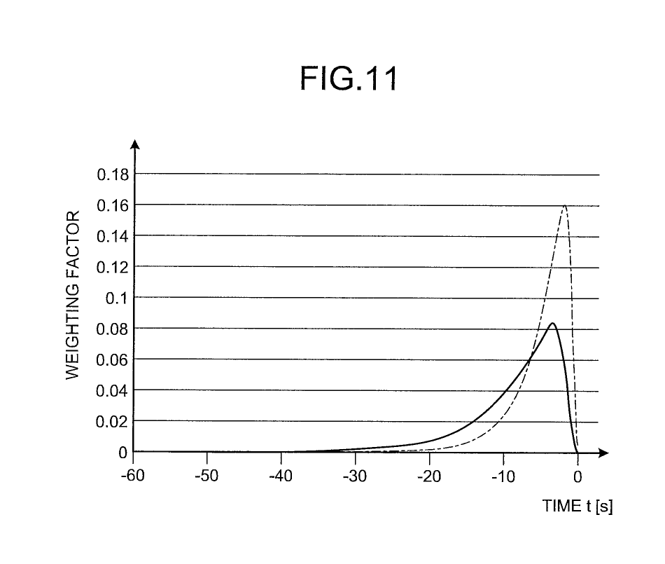

FIG. 10 is a diagram illustrating the calculation example of Step S17. In the calculation of Mathematical Formula (1), values for the sampling intervals Δt are used, but in FIG. 11, for the convenience of description, only the values of t=0 s, −10 s, −20 s, −30 s, and −40 s are described.

In the example of FIG. 10, the integration time Period_max is 40 s, the atmosphere temperature Tₐₜₘₒₛₚₕₑᵣₑ is set to 40° C., and the sampling interval Δt is set to 0.05 s. Weighting factors α(t) at t=0 s, −10 s, −20 s, −30 s, and −40 s are set to “0”, “0.04”, “0.01”, “0.007”, and “0.0005”, respectively (refer to the solid line in FIG. 11).

In addition, in the example of FIG. 10, when the current time is 150 s (for example, a time from the time when the foot switch 4 is turned on), the heater temperatures Tₕₑₐₜₑᵣ calculated at the current time (t=0 s) and the times (t=−10 s, −20 s, −30 s, and −40 s) of 10 s, 20 s, 30 s, and 40 s before the current time are set to 150° C., 200° C., 300° C., 200° C., and 100° C., respectively.

Then, when the current time is 150 s, in Step S17, the outer surface temperature T_{surface} is calculated (estimated) by using Mathematical Formula (1) as described below.

That is, based on Mathematical Formula (1), the temperature estimation unit 338 calculates each difference between the atmosphere temperature Tₐₜₘₒₛₚₕₑᵣₑ and the heater temperature Tₕₑₐₜₑᵣ(t) at every sampling interval Δt in the integration time Period_max, multiplies each differences with the weighting factor α(t) for the corresponding time t, performs integration, and adds up the integrated value to the atmosphere temperature Tₐₜₘₒₛₚₕₑᵣₑ to calculate (estimate) the outer surface temperature T_{surface}.

Specifically, α(t)(Tₕₑₐₜₑᵣ(t)−Tₐₜₘₒₛₚₕₑᵣₑ)Δt at t=0 s is 0× (150−40)×0.05=0. In addition, α(t)(Tₕₑₐₜₑᵣ(t)−Tₐₜₘₒₛₚₕₑᵣₑ)Δt at t=−10 s is 0.04× (200−40)×0.05=0.32. In addition, α(t)(Tₕₑₐₜₑᵣ(t)−Tₐₜₘₒₛₚₕₑᵣₑ)Δt at t=−20 s is 0.01× (300−40)×0.05=0.13. In addition, α(t)(Tₕₑₐₜₑᵣ (t)−Tₐₜₘₒₛₚₕₑᵣₑ)Δt at t=−30 s is 0.007× (200−40)×0.05=0.056. In addition, α(t)(Tₕₑₐₜₑᵣ(t)−Tₐₜₘₒₛₚₕₑᵣₑ)Δt at t=−40 s is 0.0005×(100-40)×0.05=0.0015.

Then, the values α(t)(Tₕₑₐₜₑᵣ (t)−Tₐₜₘₒₛₚₕₑᵣₑ)Δt at t=0 s, 0.05 s, 0.1 s, 0.15 s, . . . , and 40 s in the integration time Period_max are added up, and the atmosphere temperature Tₐₜₘₒₛₚₕₑᵣₑ (=40° C.) is added to the added-up value, so that the outer surface temperature T_{surface} when the current time is 150 s is calculated (estimated).

In addition, in the example of FIG. 10, the heater temperature calculated when the current time is 160 s (t=0 s) is set to 130° C. When the current time is 160 s, since 10 s has elapsed from the above-described current time of 150 s, the heater temperatures Tₕₑₐₜₑᵣ calculated at the times (t=−10 s, −20 s, −30 s, and −40 s) of 10 s, 20 s, 30 s, and 40 s before the current time (160 s) are the same as the heater temperatures Tₕₑₐₜₑᵣ at t=0 s, −10 s, −20 s, and −30 s when the above-described current time is 150 s, respectively.

Then, when the current time is 160 s, the outer surface temperature T_{surface} is calculated (estimated) by using Mathematical Formula (1) in Step S17 as described below.

Specifically, α(t)(Tₕₑₐₜₑᵣ(t)−Tₐₜₘₒₛₚₕₑᵣₑ)Δt at t=0 s is 0× (130−40)×0.05=0. In addition, α(t)(Tₕₑₐₜₑᵣ(t)−Tₐₜₘₒₛₚₕₑᵣₑ)Δt at t=−10 s is 0.04× (150−40)×0.05=0.22. In addition, α(t)(Tₕₑₐₜₑᵣ(t)−Tₐₜₘₒₛₚₕₑᵣₑ)Δt at t=−20 s is 0.01× (200−40)×0.05=0.08. In addition, α(t)(Tₕₑₐₜₑᵣ (t)−Tₐₜₘₒₛₚₕₑᵣₑ)Δt at t=−30 s is 0.007× (300−40)×0.05=0.091. In addition, α(t)(Tₕₑₐₜₑᵣ(t)−Tₐₜₘₒₛₚₕₑᵣₑ)Δt at t=−40 s is 0.0005× (200−40)×0.05=0.004.

Then, the values α(t)(Tₕₑₐₜₑᵣ(t)−Tₐₜₘₒₛₚₕₑᵣₑ)Δt at t=0 s, 0.05 s, 0.1 s, 0.15 s, and 40 s in the integration time Period_max are added up, and the atmosphere temperature Tₐₜₘₒₛₚₕₑᵣₑ (=40° C.) is added to the added-up value, so that the outer surface temperature T_{surface} when the current time is 160 s is calculated (estimated).

In addition, in the example of FIG. 10, the heater temperature calculated when the current time is 170 s (t=0 s) is set to 170° C. When the current time is 170 s, since 10 s has elapsed from the above-described current time of 160 s, the heater temperatures Tₕₑₐₜₑᵣ calculated at the times (t=−10 s, −20 s, −30 s, and −40 s) of 10 s, 20 s, 30 s, and 40 s before the current time (170 s) are the same as the heater temperatures Tₕₑₐₜₑᵣ at t=0 s, −10 s, −20 s, and −30 s when the above-described current time is 160 s, respectively.

Then, when the current time is 170 s, the outer surface temperature T_{surface} is calculated (estimated) by using Mathematical Formula (1) in Step S17 as described below.

Specifically, α(t)(Tₕₑₐₜₑᵣ (t)−Tₐₜₘₒₛₚₕₑᵣₑ)Δt at t=0 s is 0×(170-40)×0.05=0. In addition, α(t)(Tₕₑₐₜₑᵣ (t)−Tₐₜₘₒₛₚₕₑᵣₑ)Δt at t=−10 s is 0.04×(130−40)×0.05=0.18. In addition, α(t)(Tₕₑₐₜₑᵣ (t)−Tₐₜₘₒₛₚₕₑᵣₑ)Δt at t=−20 s is 0.01×(150−40)×0.05=0.055. In addition, α(t)(Tₕₑₐₜₑᵣ (t)−Tₐₜₘₒₛₚₕₑᵣₑ)Δt at t=−30 s is 0.007×(200−40)×0.05=0.056. In addition, α(t)(Tₕₑₐₜₑᵣ(t)−Tₐₜₘₒₛₚₕₑᵣₑ)Δt at t=−40 s is 0.0005×(300−40)×0.05=0.0065.

Then, the values α (t)(Tₕₑₐₜₑᵣ (t)−Tₐₜₘₒₛₚₕₑᵣₑ)Δt at t=0 s, 0.05 s, 0.1 s, 0.15 s, . . . , and 40 s in the integration time Period_max are added up, and the atmosphere temperature Tₐₜₘₒₛₚₕₑᵣₑ (=40° C.) is added to the added-up value, so that the outer surface temperature T_{surface} when the current time is 170 s is calculated (estimated).

Method of Calculating Weighting Factor α(t)

Next, a method of calculating the weighting factor α(t) used in the above Steps S16 and S17 will be described.

FIG. 11 is a diagram illustrating an example of the weighting factor α(t) used in Steps S16 and S17. Specifically, in FIG. 11, the horizontal axis indicates a time t going back from the current time to a past (the time t at the current time is 0 s and the time t before the current time is a negative value), and the vertical axis indicates a weighting factor. In addition, the weighting factor α(t) indicated by the solid line in FIG. 11 is the weighting factor α(t) used in the calculation example of the outer surface temperature T_{surface} in FIG. 10.

As described above, the weighting factor α(t) used in Steps S16 and S17 is calculated by experiment in advance and stored in the second memory 337.

In the experiment, in the state where the first holding member 8 and the energy generator 10 to be actually used are assembled, the energy generator 10 (wiring pattern 132) is electrically conducted so that the heater temperature Tₕₑₐₜₑᵣ becomes a constant temperature. Then, the weighting factor α(t) is calculated by actually measuring the outer surface temperature T_{surface} by a temperature sensor (not illustrated) at every sampling interval Δt from the start of electrical conduction to the energy generator 10, inserting the actually measured outer surface temperature T_{surface}, the heater temperature Tₕₑₐₜₑᵣ which is set to a constant temperature, and the atmosphere temperature Tₐₜₘₒₛₚₕₑᵣₑ into Mathematical Formula (1), and performing back calculation.

As the tendency of the weighting factor α(t), as indicated by the solid line in FIG. 11, there is a peak immediately before t=0 s at the current time (t=−3.4 s in the example of FIG. 11), and thus, the time t approaches 0 as the time goes from the peak towards the past.

In the second embodiment, the time t which is a value (weighting factor) of equal to or lower than 1/100 of the peak value is set as the integration time Period_max. Specifically, the weighting factor at t=−40 s is 0.0005, which is equal to or lower than 1/100 of the peak value (0.084 at t=−3.4 s). Therefore, in the calculation example of the outer surface temperature T_{surface} in FIG. 10, the integration time Period_max is set to 40 s.

The weighting factor α(t) varies depending on the configurations and thermal properties of the first holding member 8 and the energy generator 10. For example, as can be seen by comparing a case where the thermal conductivity of the first holding member 8 is high (the one-dot dashed line in FIG. 11) and a case where the thermal conductivity is low (the solid line in FIG. 11), as the thermal conductivity increases, the peak value becomes increased, and thus, the time t when the value reaches the peak value also approaches the current time (t=0 s).

According to the treatment system 1A according to the second embodiment, in addition to the effects of the first embodiment described above, the following effects may be achieved.

In the treatment system 1A according to the second embodiment, the outer surface temperature T_{surface} is estimated based on the atmosphere temperature Tₐₜₘₒₛₚₕₑᵣₑ and the heater temperature Tₕₑₐₜₑᵣ without actually measuring the outer surface temperature.

Therefore, there is no need to provide the temperature sensor 11, and thus, the structure of the treatment tool 2A may be simplified.

Particularly, since the outer surface temperature T_{surface} is estimated by Mathematical Formula (1) using the weighting factor α(t) calculated in advance by experiment, the outer surface temperature T_{surface} may be estimated with high accuracy.

### Third Embodiment

Next, a third embodiment will be described.

In the following description, the same configurations as those of the second embodiment described above are denoted by the same reference numerals, and the detailed description thereof will be omitted or simplified.

In the treatment system according to the third embodiment described above is different from the treatment system 1A described in the second embodiment in terms of a method of calculating (estimating) the outer surface temperature.

Hereinafter, the configuration of the treatment system according to the third embodiment and the operations of the control device constituting the treatment system will be sequentially described.

Configuration of Treatment System

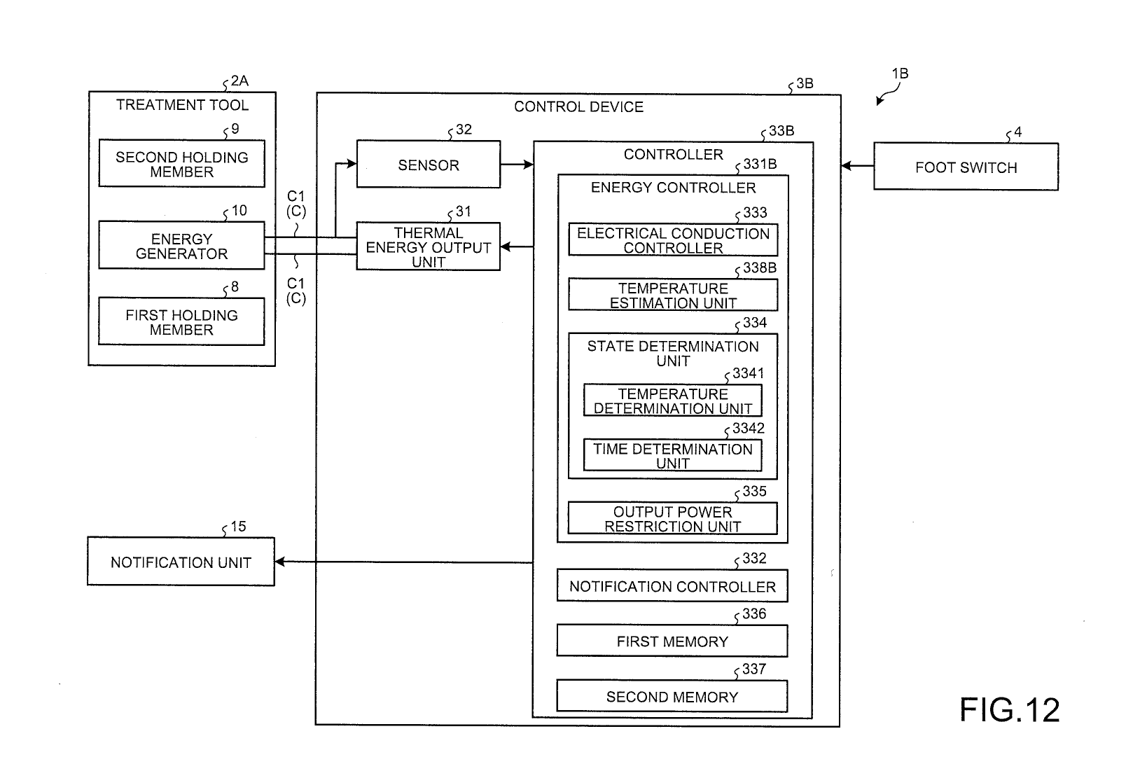

FIG. 12 is a block diagram illustrating a control device 3B constituting a treatment system 1B according to the third embodiment.

As illustrated in FIG. 12, the treatment system 1B adopts the control device 3B (controller 33B (energy controller 331B)) in which a temperature estimation unit 338B having functions different from those of the temperature estimation unit 338 is added instead of the control device 3A to the treatment system 1A (FIG. 8) described in the second embodiment. In addition, the first memories 336 and 337 according to the third embodiment are different from the first memories 336 and 337 described in the second embodiment in terms of the information to be stored.

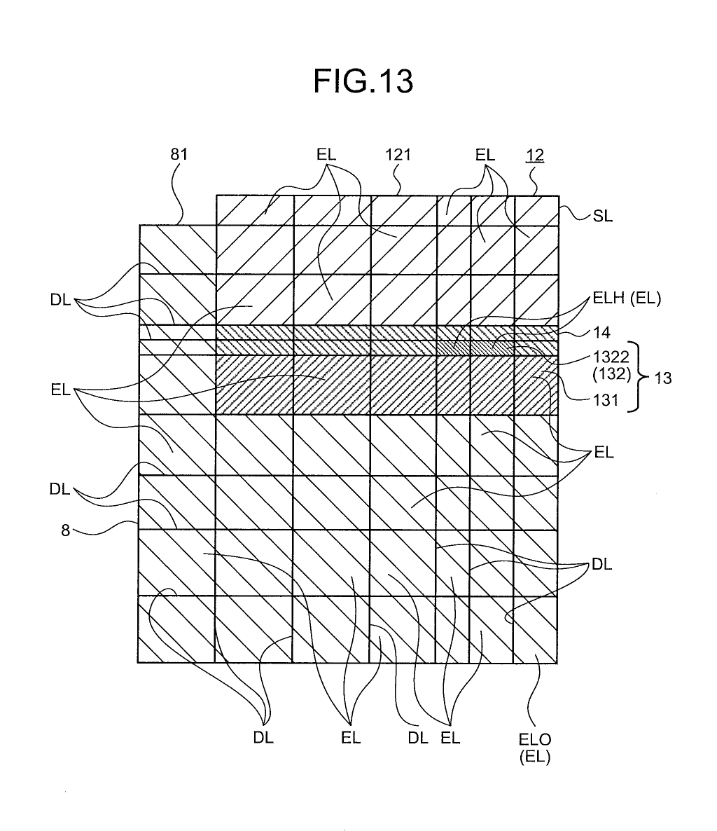

The first memory 336 according to the third embodiment stores the numerical calculation result of each element EL (refer to FIG. 13) calculated (estimated) by the temperature estimation unit 338B.

The second memory 337 according to the third embodiment stores a control program executed by the controller 33B and an assumed atmosphere temperature outside the treatment system 1B (assumed to be used in a living body, and thus, about 37° C. to 40° C.), and thermal diffusivity D of each component constituting the first holding member 8 and the energy generator 10.

The temperature estimation unit 338B calculates (estimates) the outer surface temperature by using a predetermined analysis model.

FIG. 13 is a diagram illustrating an example of an analysis model used in the temperature estimation unit 338B. Specifically, FIG. 13 is a cross-sectional view corresponding to FIG. 5.

As illustrated in FIG. 13, in the analysis model, used is a cross sectional view obtained by cutting the first holding member 8 and the energy generator 10 at the cut surface along the width direction of the first holding member 8, assuming that there is no exchange of heat by the symmetrical line SL passing through the center position in the width direction, and further cutting the first holding member 8 and the energy generator 10 in half by the symmetrical line SL. In addition, in the analysis model, the first holding member 8 and the energy generator 10 are divided into a plurality of elements EL by a plurality of division lines DL passing through the boundary lines or the like of the respective components of the first holding member 8 and the energy generator 10.

Then, the temperature estimation unit 338B numerically calculates the temperature of each element EL for each element EL according to the configurations of the first holding member 8 and the energy generator 10 and the unsteady heat conduction equation of the following Mathematical Formula (2) led by thermal properties. As a result, the temperature estimation unit 338B adopts the temperature of the outer element ELO (FIG. 13) located on the outer surface among the elements EL as the outer surface temperature.

$$\frac{\partial T}{\partial t} = D ( \frac{{\partial }^{2} T}{\partial {x}^{2}} + \frac{{\partial }^{2} T}{\partial {y}^{2}} ) \qquad ( 2 )$$

In Mathematical Formula (2), D is thermal diffusivity of each member constituting the first holding member 8 and the energy generator 10.

Operation of Control Device

Next, the operations of the control device 3B described above will be described.

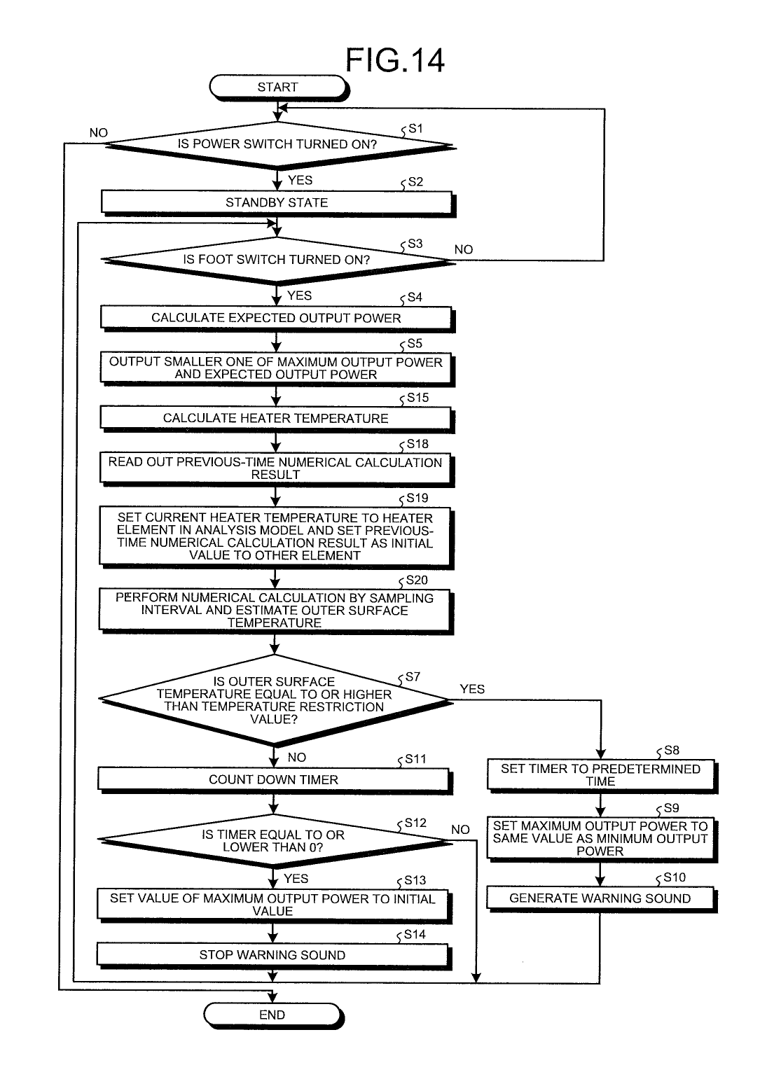

FIG. 14 is a flowchart illustrating the operations of the control device 3B.

As illustrated in FIG. 14, the operations of the control device 3B according to the third embodiment are different from the operations (FIG. 9) of the control device 3A described in the second embodiment only in that Steps S18 to S20 are added instead of Steps S16 and S17. Therefore, only Steps S18 to S20 will be described below.

Step S18 is executed after Step S15.

Specifically, in Step S18, the temperature estimation unit 338B reads out the previous-time numerical calculation result (temperature) of each of the elements EL stored in the first memory 336. In addition, since the previous-time numerical calculation result does not exist at the time of activation (the first time of this control flow), the temperature estimation unit 338B reads out the atmosphere temperature stored in the second memory 337.

After Step S18, the temperature estimation unit 338B sets the current heater temperature calculated in Step S15 to the heater element ELH (FIG. 13) corresponding to the heat generation pattern 1322 among the elements EL and sets the previous-time numerical calculation result for another element EL as an initial value (Step S19). Since the previous-time numerical calculation result does not exist at the time of activation (the first time of this control flow), the atmosphere temperature read out in Step S18 for another element EL is set as the initial value.

After Step S19, the temperature estimation unit 338B performs numerical calculation for each element EL by a sampling time (for example, 0.05 s) according to the unsteady heat conduction equation of Mathematical Formula (2) and calculates (estimates) the temperature of the outer element ELO as the outer surface temperature (Step S20). Then, the temperature estimation unit 338B stores (overwrites and saves) the numerical calculation result for each element EL in the first memory 336. After that, the control device 3B proceeds to Step S7.

Even in the case of adopting the configuration of calculating (estimating) the outer surface temperature by numerical calculation using the unsteady heat conduction equation (Mathematical Formula (2)) as in the third embodiment described above, it is possible to obtain the same effect as that of the second embodiment.

### Other Embodiments

Up to this point, the modes for carrying out the present disclosure have been described, but the present disclosure is not limited only by the first to third embodiments described above.

In the first to third embodiments described above, the treatment tools 2 and 2A are configured to apply thermal energy to the living tissue, but the present disclosure is not limited thereto, and the treatment tool may be configured to apply high-frequency energy or ultrasonic energy.

In the first to third embodiments described above, the configuration where the energy generator 10 is provided only to the first holding member 8 is adopted, but the present disclosure is not limited thereto, and the configuration where the energy generator 10 may also be provided to the second holding member 9 may be adopted.

In the first to third embodiments described above, the control flow is not limited to the flows illustrated in FIGS. 7, 9, and 14, and the order thereof may be changed within a range where contradiction does not occur.

For example, it may be possible to adopt a configuration where Steps S10 and S14 are omitted (the notification unit 15 and the notification controller 332 are omitted) and only the output power restriction (Steps S9 and S13) is performed based on the determination result of the state determination unit 334. On the contrary, it may be possible to adopt a configuration where Steps S9 and S13 are omitted (the output power restriction unit 335 is omitted) and only the warning sound generation (Steps S10 and S14) is performed based on the determination result of the state determination unit 334.

In addition, for example, in the output power restriction in Step S9, the output value (power value) supplied (electrically conducted) to the energy generator 10 is restricted to the minimum output power (for example, 0.1 W), but the present disclosure is not limited there to, and the supply of the output value (power value) to the energy generator 10 may be stopped.

In addition, for example, in Step S10, the generated warning sound need not be constant, and the warning sound may be changed to a large sound or a high sound as the outer surface temperature is increased. In addition, when the notification unit 15 is configured with a display, for example, the display may be configured so that a green circle is displayed for a case where the outer surface temperature is equal to or lower than 80° C., a yellow circle is displayed for a case where the outer surface temperature is within a range of 80° C. to 100° C., a red circle is displayed for a case where the outer surface temperature is equal to or higher than 100° C., or the like. In addition, for example, a configuration may be provided where, the higher the outer surface temperature is, the higher the blinking speed of the warning display is. Furthermore, it is possible to combine the warning sound and the warning display.

Although the two-dimensional unsteady heat conduction equation (Mathematical Formula (2)) is used in the third embodiment described above, the present disclosure is not limited thereto, and a one-dimensional or three-dimensional unsteady heat conduction equation may be used.

In the first to third embodiments described above, each of the control devices 3, 3A, and 3B is provided outside each of the treatment tools 2 and 2A, but the present disclosure is not limited thereto, and the configuration may be adopted where each of the control devices 3, 3A, and 3B may be provided inside each of the treatment tools 2 and 2A (for example, inside the handle 5).

A treatment system and a treatment tool according to the present disclosure have an effect of being able to avoid an unintended action on a living tissue at a portion other than a treatment surface.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment system comprising:
a probe comprising
an energy generator provided in the probe and configured to generate energy, and
a treatment surface provided at a distal end of the probe and configured to apply the energy to a living tissue; and
a controller comprising a hardware, the controller being configured to:
acquire a temperature of an outer surface of the probe other than the treatment surface;
sequentially store temperatures of the energy generator in association with times when the temperatures of the energy generator are acquired;
store a plurality of weighting factors calculated by experiment in advance in association with times going back from a current time to a past time; and
multiply the sequentially stored temperatures of the energy generator and the plurality of weighting factors for the corresponding times, perform integration, and estimate the temperature of the outer surface.

2. The treatment system according to claim 1, wherein the controller is configured to store an atmosphere temperature which is an assumed temperature in a body cavity, and calculate differences between the atmosphere temperature and the sequentially stored temperatures of the energy generator, multiply the differences and the plurality of weighting factors for the corresponding times, perform integration, and add up the integrated value to the atmosphere temperature to estimate the temperature of the outer surface.

3. The treatment system according to claim 1, wherein the controller is configured to restrict an amount of the energy generated in the energy generator when the temperature of the outer surface is equal to or higher than a threshold value.

4. The treatment system according to claim 1, wherein the controller is configured to notify predetermined information, and operate the notification unit when the temperature of the outer surface is equal to or higher than a threshold value.
